(19) United States
(12) Patent Application Publication
(10) Pub. No.: US 2026/0340775 A1
(43) Pub. Date: Oct. 29, 2026

(54) Transparent Arterial Layer Separation in Cardiac Angiography with Self-Supervised Deep Learning

(71) Applicant: Northwestern University, Evanston, IL (US)

(72) Inventors: Syed Hasib Akhter Faruqui, Chicago, IL (US); Donald Robinson Cantrell, Chicago, IL (US); Sameer Ahmad Ansari, Chicago, IL (US); Leon Cho, Chicago, IL (US); Chaochao Zhou, Chicago, IL (US)

(21) Appl. No.: 19/485,829

(22) PCT Filed: May 20, 2024
(86) PCT No.: PCT/US2024/030247

§ 371 Date: Nov. 19, 2025

Related U.S. Application Data
(60) Provisional application No. 63/467,805, filed on May 19, 2023.

(51) Int. Cl. G06T 5/50; G06T 5/60; G06T 7/194; G06T 7/246

(52) U.S. Cl. CPC G06T 5/50; G06T 5/60; G06T 7/194; G06T 7/246; G06T 2207/10116; G06T 2207/20084; G06T 2207/20224; G06T 2207/30021; G06T 2207/30048; G06T 2207/30101

(57) ABSTRACT

A system and method of catheter angiography utilizing a deep neural network-based digital subtraction angiogram (DSA) algorithm for accurately recovering vessel structures and intensity information from cardiac angiography acquisitions including the steps of receiving X-ray coronary angiography (XCA) images from an individual, generating a mask of vessel layers from the XCA images, extracting vessel layers from a background image and a foreground image in which the background image and the foreground image comprise voluntary motion, respiratory motion, cardiac motion, or other artifact, and generating a final angiographic image comprising the extracted vessel layers.

### CROSS REFERENCE TO RELATED APPLICATION

This application claims priority to U.S. Provisional Patent Application No. 63/467,805 filed on May 19, 2023, which is hereby incorporated by reference herein.

### TECHNICAL FIELD

This disclosure relates to the field of catheter angiography imaging techniques that allow physicians to visualize vascular disorders with unparalleled spatial and temporal resolution.

### BACKGROUND

Catheter angiography is a minimally invasive imaging technique that allows physicians to visualize vascular disorders and is considered the gold standard for diagnosing and characterizing vascular diseases throughout the body, and it provides the foundation for endovascular interventions to treat numerous devastating pathologies, including stroke, cerebral aneurysms, arteriovenous malformations, and myocardial infarctions. Catheter angiograms are performed by inserting a small catheter into an artery (vessel), injecting iodinated contrast through the catheter, and recording a series of fluoroscopic (X-ray) images as the contrast traverses the vasculature. However, superimposed fluoroscopic densities from the bones and soft tissues obscure the evaluation of the vessels. Visualization of the vasculature during cardiac angiography is a particular challenge, as the heart is continuously beating, which causes severe image degradation when using current digital subtraction angiogram (DSA) techniques. As such, an improved system and method for accurately recovering vessel structures and intensity information from cardiac angiography acquisitions would be advantageous.

### SUMMARY

This Summary introduces a selection of concepts relating to this technology in a simplified form as a prelude to the Detailed Description that follows. This Summary is not intended to identify key or essential features.

A deep neural network-based DSA algorithm for accurately recovering vessel structures and intensity information from cardiac angiography acquisitions is disclosed herein. See also, PCT/US21/37936, Maskless 2D/3D Artificial Subtraction Angiography, filed Jun. 17, 2021, and incorporated herein by reference in its entirety for all purposes. The proposed systems and methods disclosed herein may utilize all the frames of the cardiac angiograms and may find the inter-frame spatio-temporal connectivity in the complex and noisy backgrounds to separate and extract the vessels from the background (vessel-masked). The background image may contain obstructions, e.g., the beating heart, bones, and other artifacts. The proposed model may be evaluated on real X-ray angiography data (n=200). Experimental results disclosed herein show the superiority of the disclosed system and method over conventional systems and methods.

In one aspect, a system and method of catheter angiography imaging via a deep neural network-based digital subtraction angiography (DSA) algorithm is disclosed herein in which each of the following steps may be performed by a computing device comprising at least one processor, a communication interface, and memory, including receiving X-ray coronary angiography (XCA) images from an individual, generating a mask of vessel layers from the XCA images, calculating an optical flow comprising a forward flow and a backward flow, determining uncertain areas of dense optical flow using the forward flow and the backward flow, generating a confidence map using the uncertain areas of dense optical flow in which the confidence map identifies areas where the optical flow calculation may be less confident, extracting vessel layers from a background image and a foreground image, and generating a final angiographic image comprising the extracted vessel layers. In some examples, the forward flow may be used to extract the vessel layers from the background image and the foreground image. In other examples, the confidence map may also be used to extract the vessel layers from the background image and the foreground image. In certain examples, the background image and the foreground image may include voluntary motion, respiratory motion, cardiac motion, or other artifact. In still other examples, the XCA images may be received in real-time or near real-time, and/or the final angiographic image may be generated in real-time or near real-time.

In another aspect, method of digital subtraction angiography (DSA) imaging is disclosed herein in which each of the foregoing steps may be performed by a computing device comprising at least one processor, a communication interface, and memory, including receiving X-ray coronary angiography (XCA) images from an individual, generating a mask of vessel layers from the XCA images, extracting vessel layers from a background image and a foreground image in which the background image and the foreground image comprise voluntary motion, respiratory motion, cardiac motion, or other artifact, and generating a final angiographic image comprising the extracted vessel layers.

In other examples, the XCA images may be received generated in real-time or near real-time and/or the final angiographic image may be generated in real-time or near real-time. In another example, the method disclosed herein may further include calculating an optical flow including a forward flow and a backward flow, and the forward flow may used to extract the vessel layers from the background image and the foreground image. In yet another example, the forward flow and the backward flow may include vessel region structure data only. In one example, the method disclosed herein may further include determining uncertain areas of dense optical flow using the forward flow and/or the backward flow. In still other examples, the method disclosed herein may further include generating a confidence map using the uncertain areas of dense optical flow. In certain examples, the confidence map may identify areas where the optical flow calculation may be less confident. In some examples, the confidence map may be used to extract the vessel layers from the background image and the foreground image. In other examples, the method disclosed herein may further include registering the background image as stationary. In another example, the method disclosed herein may further include registering the foreground image as that of a slow cardiac motion prior to extracting the vessel layers from the background image and the foreground image. In yet another example, the received XCA images may be from an individual in a remote location. In still another example, the received XCA images may be from a historical database. In still other examples, the XCA images may be received in real-time or near real-time, and/or the final angiographic image may be generated in real-time or near real-time.

In other aspects, a system for generating an angiographic image may include memory storing computer-readable instructions causing the system to receive X-ray coronary angiography (XCA) images from an individual, generate a mask of vessel layers from the XCA images, extract the vessel layers from a background image and a foreground image in which the background image and the foreground image comprise voluntary motion, respiratory motion, cardiac motion, or other artifact, and generate a final angiographic image comprising the extracted vessel layers.

In some examples, the XCA images may be received and the final angiographic image is generated in real-time or near real-time. In still other examples, the system disclosed herein may further include the step of calculating, via an optical flow generator, an optical flow including a forward flow and a backward flow, and the forward flow may be used to extract the vessel layers from the background image and the foreground image. In one example, the forward flow and the backward flow may include vessel region structure data only. In another example, the system disclosed herein may further include the step of determining, via the optical flow generator, uncertain areas of dense optical flow using the forward flow and the backward flow. In yet another example, the system disclosed herein may further include the step of generating, via a confidence generator, a confidence map using the uncertain areas of dense optical flow. In certain examples, the confidence map may identify areas where the optical flow calculation may be less confident. In another example, the confidence map may be used to extract the vessel layers from the background image and the foreground image. In yet another example, the received XCA images may be from an individual in a remote location. In still another example, the received XCA images may be from a historical database. In still other examples, the XCA images may be received in real-time or near real-time, and/or the final angiographic image may be generated in real-time or near real-time.

These and other features, advantages, and objects of the present disclosure will be further understood and appreciated by those skilled in the art by reference to the following specification, claims, and appended drawings, where various embodiments of the design illustrate how concepts of this disclosure may be used.

### BRIEF DESCRIPTION OF THE DRAWINGS

A more complete understanding of features described herein and advantages thereof may be acquired by referring to the following description in consideration of the accompanying drawings, in which like reference numbers indicate like features. The patent or application file contains at least one drawing executed in color. Copies of this patent or patent application publication with color drawing(s) will be provided by the Office upon request and payment of the necessary fee.

FIG. 1A illustrates an example of a deep learning neural network architecture in which one or more aspects described herein may be implemented.

FIG. 1B illustrates an example computing device in accordance with one or more aspects described herein.

FIG. 1C schematically illustrates layer separation using input X-ray coronary angiography (XCA) images, a rough mask, forward optical flow, a confidence map, and motion registration information to separate the forward and backward layers as disclosed herein.

FIG. 1D illustrates XCA images represented as a combination of static background and dynamic foreground in accordance with Equation (2).

FIGS. 2A-D illustrate masks generated by different methods as disclosed herein; (a) Original XCA Image, (b) VAE Generated Mask, (c) Robust-PCA Generated Mask, and (d) Hand-drawn Mask (with the major arteries are marked in this case).

FIG. 3 illustrates generating optical flow as disclosed herein. Forward flow may be generated by passing the frame sequence to the RAFT algorithm in the natural order, and backward flow may be generated by passing them in reverse order.

FIGS. 4A-4B illustrate (a) Mask Generator: A variation of β-VAE may be utilized to generate the static backgrounds and masks. A ResNet50 backend may be used for extracting features from input XCA, (b) An example of the generated mask using the model. The far right image shows the mask generated given the input XCA neural tuning of computed tomography (NeTCT) in DiffXP.

FIG. 5 illustrates Separated Layers: Example 1: (a-g) original XCA sequence, (h-n) separated background layer, and (o-u) separated foreground layer; Example 2: (v-ab) original XCA sequence, (ac-ai) separated background layer, and (aj-ap) separated foreground layer.

FIG. 6A-6D illustrate post-processing of the separated foreground (dynamic) layer.

FIG. 7 illustrates Separated Layer (Failure Case): (a-g) Original XCA sequence, (h-n) Separated Background Layer; failure to capture the motion of the spine from t=1 to t=61 and the bottom right corner of the spine is missing pixel information in the background reconstruction, and (o-u) Separated Foreground Layer; captured motion artifacts from the motion of the background (i.e., spine) from t=1 to t=61.

### DETAILED DESCRIPTION

In the following description of the various embodiments, reference is made to the accompanying drawings identified above and which form a part hereof, and in which is shown by way of illustration various embodiments in which features described herein may be practiced. It is to be understood that other embodiments may be utilized and structural and functional modifications may be made without departing from the scope described herein. Various features are capable of other embodiments and of being practiced or being carried out in various different ways.

The disclosed subject matter may be further described using definitions and terminology as follows. The definitions and terminology used herein are for the purpose of describing particular embodiments only, and are not intended to be limiting.

As used in this specification and the claims, the singular forms “a,” “an,” and “the” include plural forms unless the context clearly dictates otherwise. For example, the term “patient” should be interpreted to mean “one or more patients” and unless the context clearly dictates otherwise. As used herein, the term “plurality” means “two or more.”

As used herein, “about”, “substantially,” and “significant” will be understood by persons of ordinary skill in the art and will vary to some extent on the context in which they are used. If there are uses of the term that are not clear to persons of ordinary skill in the art given the context in which it is used, “about” will mean up to plus or minus 10% of the particular term and “substantially” and “significant” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into subranges as discussed above.

A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

In the present disclosure, the terms “patient” and “individual” may refer to a human or non-human being. The terms “patient” and “individual” may be used interchangeably throughout the present disclosure. The terms “algorithm” “model” “equation” “system” and “method” may be used interchangeably throughout the present disclosure.

In the present disclosure, the terms “real-time” and “near real-time” may refer to processing that requires a continual input, constant processing, and steady output of data which may occur over the course of seconds or milliseconds, fractions of seconds, or minutes. The terms “real-time” and “near real-time” may be used interchangeably throughout the present disclosure.

Catheter angiography is performed by inserting a small catheter into the arteries of the body, injecting iodinated contrast through the catheter, and recording a series of fluoroscopic images as the contrast traverses the vasculature. However, superimposed fluoroscopic densities from the bone and soft tissues obscure evaluation of the vessels. For decades, angiographers have utilized DSA to better visualize the vasculature. In this technique, a fluoroscopic image mask is acquired prior to contrast injection, and the mask is subsequently subtracted from image frames recorded after contrast injection. In ideal conditions, DSA will provide an image of the vessels alone, unobscured by superimposed bone and soft tissue densities. This concept has been further extended to 3D rotational angiography, in which a full 3D acquisition is obtained before and after contrast injection to improve visualization of the vasculature.

DSA, however, is heavily affected by motion that occurs between the acquisition of the mask frame and subsequent images. Thus, voluntary, respiratory, or cardiac motion can all degrade vascular imaging with the Digital Subtraction technique. Furthermore, the acquisition of a mask frame increases radiation exposure to the patient and staff, particularly during rotational angiography, which requires many X-ray projections to compute 3D reconstructions, substantially increasing the radiation dose.

A deep learning algorithm disclosed herein may perform both vessel segmentation and computational isolation of vascular contrast enhancement on raw angiographic images to mitigate or overcome the degrading effects of motion and to minimize the radiation dose.

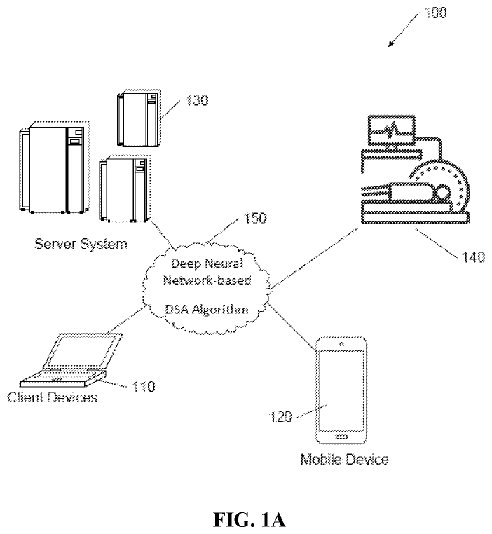

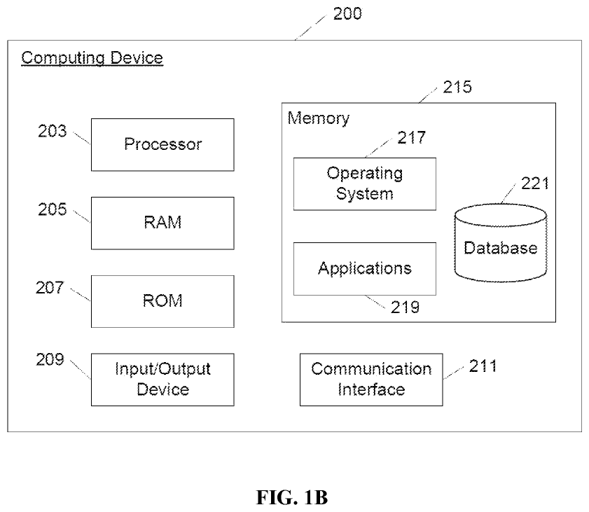

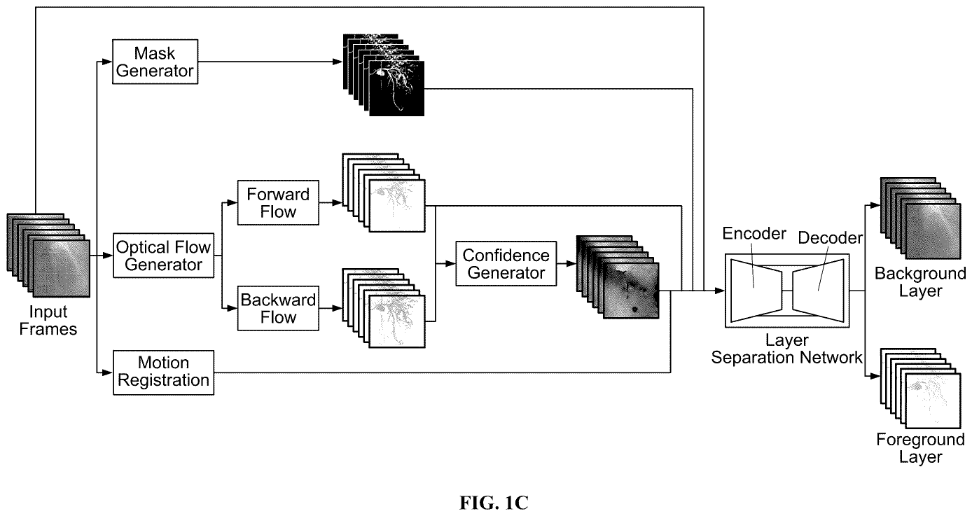

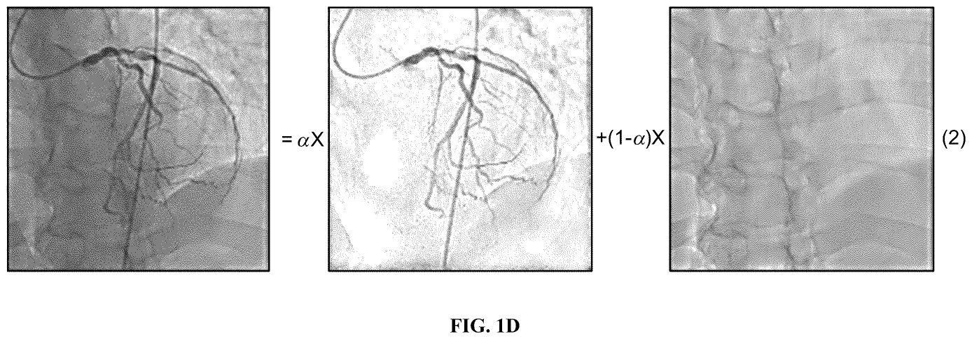

FIG. 1A illustrates an operating environment 100 in accordance with an embodiment of the invention. The operating environment 100 may include at least one client device 110, and/or at least one mobile device 120, and/or at least one server system 130, and at least one angiographic imaging system 140 in communication via a deep neural network-based DSA algorithm 150. It will be appreciated that the network connections shown are illustrative and any means of establishing a communications link between the computers may be used. The existence of any of various network protocols such as TCP/IP, Ethernet, FTP, HTTP and the like, and of various wireless communication technologies such as GSM, CDMA, WiFi, and LTE, is presumed, and the various computing devices described herein may be configured to communicate using any of these network protocols or technologies. Any of the devices and systems described herein may be implemented, in whole or in part, using one or more computing devices described with respect to FIG. 1B.

Client devices 110 and/or mobile device 120 may provide user interface requests to the server system 130, and/or the angiographic system 140, and/or the deep neural network-based DSA algorithm 150 as described herein. The network 150 may include a local area network (LAN), a wide area network (WAN), a wireless telecommunications network, and/or any other communication network or combination thereof.

Some or all of the data described herein may be stored using any of a variety of data storage mechanisms, such as databases. These databases may include, but are not limited to relational databases, hierarchical databases, distributed databases, in-memory databases, flat file databases, XML databases, NoSQL databases, graph databases, and/or a combination thereof. The data transferred to and from various computing devices in the operating environment 100 may include secure and sensitive data, such as confidential documents, customer personally identifiable information, and account data. It may be desirable to protect transmissions of such data using secure network protocols and encryption and/or to protect the integrity of the data when stored on the various computing devices. For example, a file-based integration scheme or a service-based integration scheme may be utilized for transmitting data between the various computing devices. Data may be transmitted using various network communication protocols. Secure data transmission protocols and/or encryption may be used in file transfers to protect the integrity of the data, for example, File Transfer Protocol (FTP), Secure File Transfer Protocol (SFTP), and/or Pretty Good Privacy (PGP) encryption. In many embodiments, one or more web services may be implemented within the various computing or mobile devices. Web services may be accessed by authorized external devices and users to support input, extraction, and manipulation of data between the various computing devices in the operating environment 100. Web services built to support a personalized display system may be cross-domain and/or cross-platform, and may be built for enterprise use. Data may be transmitted using the Secure Sockets Layer (SSL) or Transport Layer Security (TLS) protocol to provide secure connections between the computing devices. Web services may be implemented using the WS-Security standard, providing for secure SOAP messages using XML encryption. Specialized hardware may be used to provide secure web services. For example, secure network appliances may include built-in features such as hardware-accelerated SSL and HTTPS, WS-Security, and/or firewalls. Such specialized hardware may be installed and configured in the operating environment 100 in front of one or more computing or mobile devices such that any external devices may communicate directly with the specialized hardware.

Turning now to FIG. 1B, a computing device 200 in accordance with an embodiment of the invention is shown. The computing device 200 may include a processor 203 for controlling overall operation of the computing device 200 and its associated components, including RAM 205, ROM 207, input/output device 209, communication interface 211, and/or memory 215. A data bus may interconnect processor(s) 203, RAM 205, ROM 207, memory 215, I/O device 209, and/or communication interface 211. In some embodiments, computing device 200 may represent, be incorporated in, and/or include various devices such as a desktop computer, a computer server, a mobile device, such as a laptop computer, a tablet computer, a smart phone, any other types of mobile computing devices, imaging devices, and the like, and/or any other type of data processing device.

Input/output (I/O) device 209 may include a microphone, keypad, touch screen, and/or stylus through which a user of the computing device 200 may provide input, and may also include one or more of a speaker for providing audio output and a video display device for providing textual, audiovisual, and/or graphical output. Communication interface 211 may include one or more transceivers, digital signal processors, and/or additional circuitry and software for communicating via any network, wired or wireless, using any protocol as described herein. Software may be stored within memory 215 to provide instructions to processor 203 allowing computing device 200 to perform various actions. For example, memory 215 may store software used by the computing device 200, such as an operating system 217, application programs 219, and/or an associated internal database 221. The various hardware memory units in memory 215 may include volatile and nonvolatile, removable and non-removable media implemented in any method or technology for storage of information such as computer-readable instructions, data structures, program modules, or other data. Memory 215 may include one or more physical persistent memory devices and/or one or more non-persistent memory devices. Memory 215 may include, but is not limited to, random access memory (RAM) 205, read only memory (ROM) 207, electronically erasable programmable read only memory (EEPROM), flash memory or other memory technology, optical disk storage, magnetic cassettes, magnetic tape, magnetic disk storage or other magnetic storage devices, or any other medium that may be used to store the desired information and that may be accessed by processor 203.

Processor 203 may include a single central processing unit (CPU), which may be a single-core or multi-core processor, or may include multiple CPUs. Processor(s) 203 and associated components may allow the computing device 200 to execute a series of computer-readable instructions to perform some or all of the processes described herein. Although not shown in FIG. 1B, various elements within memory 215 or other components in computing device 200, may include one or more caches, for example, CPU caches used by the processor 203, page caches used by the operating system 217, disk caches of a hard drive, and/or database caches used to cache content from database 221. For embodiments including a CPU cache, the CPU cache may be used by one or more processors 203 to reduce memory latency and access time. A processor 203 may retrieve data from or write data to the CPU cache rather than reading/writing to memory 215, which may improve the speed of these operations. In some examples, a database cache may be created in which certain data from a database 221 is cached in a separate smaller database in a memory separate from the database, such as in RAM 205 or on a separate computing device. For instance, in a multi-tiered application, a database cache on an application server may reduce data retrieval and data manipulation time by not needing to communicate over a network with a back-end database server. These types of caches and others may be included in various embodiments, and may provide potential advantages in certain implementations of devices, systems, and methods described herein, such as faster response times and less dependence on network conditions when transmitting and receiving data.

Although various components of computing device 200 are described separately, functionality of the various components may be combined and/or performed by a single component and/or multiple computing devices in communication without departing from the invention.

Cardiovascular disease is one of the deadliest and most common causes of death worldwide [1, 2]. In comparison with drug therapy or invasive surgical procedures, minimally invasive interventional surgeries have emerged as a predominant clinical approach for treating coronary artery conditions [3]. However, proper planning is necessary for these procedures, and medical imaging is an essential part of this planning process. As an example, a widely adopted, minimally invasive method for cardiovascular treatment is percutaneous coronary intervention (PCI) [4]. During this procedure, contrast agents are injected into the vessels, which are imaged by X-ray coronary angiography (XCA) to help surgeons navigate the catheters [5, 6, 7]. Additionally, XCA images are also important in interventional path planning [8], stenosis automatic assessment [9, 10], coronary disease diagnosis and therapeutic evaluation [11, 12, 13], coronary three-dimensional reconstruction [7, 14], stent implantation [15], etc. Thus, accurate and rapid segmentation of vessels from XCA data is essential for the timely and effective diagnosis and intervention of cardiovascular diseases.

X-ray is typically performed by beam attenuation, which occurs in varying degrees as X-rays pass through tissues with different densities along the projection path of XCA imaging, resulting in heterogeneous blood vessels displayed in the XCA sequence [16]. These heterogeneous vessels can:
i. Overlap with various anatomical structures, including bones, lungs, and diaphragms.
ii. Overlap with peripheral irrelevant tissues.
iii. Uneven blood flow due to different blood flow rates and the Poisson-Gaussian noise [17] resulting from XCA images attenuation.
iv. Respiratory and cardiac motions also complicate the task of identifying and analyzing blood vessels.

The presence of the above-mentioned anatomical structures, including bones, lungs, and diaphragms, make it challenging for surgical teams to observe and analyze blood vessels in XCA images. Therefore, accurate and rapid segmentation of vessels from XCA data is essential for the timely and effective diagnosis and intervention of cardiovascular diseases.

Achieving an automatic diagnosis process for cardiovascular diseases requires accurate segmentation of coronary arteries, which is essential for the timely diagnosis and intervention of cardiovascular diseases. However, the limitations of angiographic images, such as low image contrast, small vessel sizes, breathing motion, and structural interference in the background, pose significant challenges to existing methods of vascular image processing, despite the development of numerous vessel extraction techniques. Therefore, the main objective of this disclosure is to propose an effective method and system for extracting vessels from X-ray angiographic image sequences, which address the limitations of existing techniques and enables the automatic diagnosis process for cardiovascular diseases.

Most vessel extraction algorithms focus on removing the background noises of XCAs. They also attempt to improve the saliency of vessels. The most simple, intuitive, and commonly used post-processing algorithm is digital subtraction angiography (DSA) [18, 19, 20]. In the DSA technique, the blood vessels are highlighted by eliminating bones, tissues, and other physical features by subtracting pre-contrast images (also referred to as masked images) from post-contrast images. The algorithm, however, is only effective when there is no motion on image sequences. Otherwise, small motion artifacts are observed on the subtracted images [16]. Thus, during a routine checkup or clinical practice, a common approach is to discard scans with excessive movements and re-collect the data again, which can be uncomfortable for patients with prior risk factors [21, 22].

To avoid the problems discussed above, researchers have proposed several methods, including methods such as registration, filtering, mass-based tracking, active contour methods, etc. [23]. Several studies have utilized registration methods to capture motion information from XCA frames that include motions. Then, by utilizing this motion information, the DSA's are improved to generate more accurate representations [24, 25, 25]. These methods, however, are computationally heavy, restrictive, and hard to implement in a real-life settings. Furthermore, these models fail to capture complex motions that are present in the XCA's [26, 27, 28, 29, 30, 31, 32]. Filter-based approaches are also popular in the medical imaging field. Filter-based methods utilize different kernels to represent the vessels with filtered responses. For example, Hessian filters can be used to generate 3D segmentation of the vessels [33, 34, 35]. These methods are also useful for retrieving and encoding geometric features (border and shape vessel structure information) [36, 37]. However, these algorithms also enhance the background structures (soft tissue, bones, spines, etc.) along with the vessels [23]. They are also dependent on the optimal scale selection major vessel radius and are highly sensitive to noise in the X-ray images [16].

Layer separation methods have become increasingly popular in recent years [29, 38]. This technique separates the different layers of anatomical structures such as blood vessels, nerves, or muscles. The technique involves segmenting each layer of the structure separately by analyzing the intensity and texture information of the image. Researchers have been using this technique to separate the lumen (inside of the vessel), the vessel wall, and the surrounding tissue [29]. As these classes of models work with a sequence of images (e.g., videos), they can capture the effect of motion within the scan frames and separate the vessel layers by removing motion artifacts. For example, in their study, Zhang et al. utilized a dense motion field in order to separate various transparent layers in XCA sequences [38]. A dynamic layer separation method incorporating dense motion estimation, uncertainty propagation, and statistical fusion was also proposed by Zhu and colleagues within a Bayesian framework [39]. On the other hand, Preston and colleagues utilized joint estimation to decompose images into different layers, and their corresponding deformations [40]. Tang et al. utilized independent component analysis (ICA) to separate vessel and background signals from XCA images. However, these methods require the subtraction of a pre-contrast mask image from later contrast images, which necessitates the registration of the two images to reduce motion artifacts. The presence of complex and dynamic backgrounds, however, may produce outliers [42, 43] in consecutive processed images [44, 45, 46]. This can limit the effectiveness of the vessel extraction methods due to image registration challenges with outliers.

Similar to Tang et al. utilizing low-dimensional representation by means of component analysis (principle component analysis, independent component analysis, robust component analysis, etc.), a set of learning techniques have been developed to analyze a sequence of XCA images [5, 16, 47]. Recently, variations of robust principal component analysis (RPCA) have gained popularity in the field of medical imaging as a low-rank sparse decomposition method. In this set of techniques, the non-stationary moving foreground layer is considered the sparse layer [16]. A combination of morphological filters and RPCA has been used to enhance the visibility of contrast-filled vessels [48], while Jin et al. incorporated spatiotemporal contiguity of vessels to develop motion coherency regularized RPCA (MCR-RPCA) [5]. Fu et al. [3] proposed total variation tensor robust principal component analysis (TV-TRPCA) where the total variation of the consecutive frames was used to separate the dynamic background. They also utilized a filtering method (Radon Filtering) to preserve shape features while extracting the vessels from the XCAs. Several other works based on RPCA are Unrolled RPCA [16], MoG-RPCA [49], ET-RPCA [50], KBR-RPCA [51], and TNN-TRPCA [52]. However, RPCA-based methods that rely on image vectorization for 2D matrix-based image sequence computation cannot naturally preserve high-dimensional imaging sensor spatial and temporal information simultaneously. Additionally, these methods are computationally expensive and require careful optimization of the hyper-parameters involved [3, 53].

In recent years, there has been an uptick of in utilizing deep learning models for vessel extraction and segmentation for XCA's. While the previously discussed models are computationally expensive and need a lot of hyperparameter tuning, deep learning models have a faster inference rate. At the same time, it is only computationally intensive during training [54]. In the field of medical image segmentation, there have also been significant improvements. Ronneberger et al. developed a 2D fully convolutional neural network (CNN) (named UNET) for segmenting biomedical images [55]. While UNET utilized 2D CNN models, Cicek et al.[56] extended UNET for volumetric segmentation replacing 2D operations with 3D operations. 3D UNet was further improved by Kayalibay et al. [57] and Isensee et al. [58] to achieve higher performance in medical image segmentation. These models opened up opportunities for better segmentation models such as UTNet [59], UNet-R [60], UNet++ [61], Swin-UNet [62], Trailing UNet [63], TransDeepLab [64], CTC-Net [65], ViT-V-Net [66], Mixed Transformer U-Net [67], Convolution-Free Transformers for image segmentation [68], TransUNet [69], MISSFormer [70], Medical Transformer [71], U-GAN [72], etc. While all these models were utilized for medical image segmentation, only a small amount of research has focused on improving DSA algorithms.

While Gao et al. [73] and Ueda et al. [74] extended deep learning models to motionless angiograms, Kimura et al. [75] developed a patch-based UNet model to extract vessel images for motion-based images. Thuy et al. [76] utilized two UNet models. The first to segment the main coronary artery and another to determine the junctions with secondary arteries. The authors then used rectangular regions to extract the secondary arteries with the second UNet model. This model was also able to capture the effects of motion between frames.

While the previously discussed models may perform reasonably at extracting vessels, historically, deep learning models are data extensive. Training these supervised models takes a large amount of labeled data [54]. Unfortunately, the manual creation of ground truth/labels for these models would require a significant amount of time from domain experts, particularly given the level of precision required for this task. Therefore, to facilitate research on developing deep learning models for extracting vessels from XCA, ground truth data may be generated given XCA data via the methods and systems as disclosed herein. This may be achieved by developing a self-supervised deep learning pipeline that utilizes low-dimensional latent space features to separate the XCA image sequences into the stationary background (e.g., bones, tissues, diaphragms, etc.) and dynamic foreground (e.g., vessel layers) images. To develop this pipeline, a video layer separation algorithm, Omnimatte [77], is utilized that may decompose a video sequence into a foreground layer (identified herein as an alpha-map, which may capture not only the vessels, but also relevant pixels that are associated with moving vessels) and background layers (e.g., anatomical structures such as blood vessels, nerves, or muscles, etc.). Further a few post-processing techniques were introduced that can improve the output of the XCA vessel layer (e.g., an RPCA-based degradation [5], a second pass at Omnimatte [77], thresholding, etc.). FIG. 1C, for example, shows a schematic overview of the method disclosed herein.

Image matting is a technique of extracting the foreground objects (in this case, the vessel layers) from the background of an image accurately. Thus, the image matting problem can be formulated as Algorithm/Equation 1:

Iᵢ=αᵢFᵢ+(1−αᵢ)Bᵢ, αᵢ∈[0,1] (1)

where Fi and Bi are the foreground and background colors of pixel i. Iᵢ represents the combined image color for the same pixel. Together, they form the final image. The parameter, αᵢ, represents the opacity that takes the value between 0 and 1. When α=1, the pixels will belong to the foreground; when α=0, the pixels will belong to the background. Thus, an XCA image can be represented as a combination of static background and dynamic foreground as illustrated in FIG. 1D (Equation 2; similar to Equation 1). This is a challenging task, especially with XCA images, as the images are mostly grayscale images, and the translucent vessel layers with thin vessels are difficult to extract accurately. Accordingly, there is a need to utilize deep learning-based models to combine multiple features (e.g., mask information, flow information, motion-registration information, etc.) extracted from raw XCAs.

In certain aspects, the present disclosure may utilize similar, but different, techniques outlined by Lu et al. [78, 77], where they proposed a deep learning model for layer separation for color images. This self-supervised learning model was trained on pervideo to reconstruct the input without observing additional (both similar or dissimilar) example videos. While utilizing this model, there were XCA video sequences that needed to meet the following assumptions:
1. The motion between frames is not too large (e.g., no panning of the X-Ray in a large area of the chest),
2. The background features had to be visible (no motion blurs) to generate motion registration features.
3. The background layer is stationary (i.e. there is only cardiac motion)

### Input Frames

As discussed above, FIG. 1C illustrates the proposed method for analyzing XCA. At its core, Omnimatte may utilize a 2D UNet, which may process the sequential XCA frames one by one. This also may utilize additional features extracted from the original XCA. The extracted features may provide Omnimatte with supplementary information that helps the model generate the separated layers with better accuracy. The additional features that need to be generated from the raw sequence of images may include (1) a rough mask of the vessel area, (2) optical flow, (3) confidence map, and (4) estimated camera motion registration information. Once the required features are extracted, the required image outputs from the Omnimatte model may be extracted. During the inference stage, Omnimatte may generate separated layers, a reconstructed refined mask, and reconstructed optical flow. The model may be restricted to only generate the alpha layers (i.e., foreground and background layers). This may be represented by Equation 3:

$$Omnimatte ( {I}_{t} , {H}_{t} , {M}_{t}^{i} , {F}_{t}^{i} ) \to {C}_{t}^{i} \qquad ( 3 )$$

where Iₜ is the sequence of input XCA images, Hₜ is the estimated camera motion registration information (homography),

$${M}_{t}^{i}$$

sequence of rough generated mask for the corresponding XCA images, and

$${F}_{t}^{i}$$

is the masked pre-computed flow field for i-th object (in this case vessel layer).

The loss function for Omnimatte model is defined as E:

[symbol]

=E_{rgb-recon}+λᵣE_{reg}+λₘEₘₐₛₖ+λ_{f}E_{flow-recon}+λ_{w}E_{alpha-warp} (4)

Where, E_{rgb-recon} reconstruction loss tied to multiple separation layer, E_{reg} is the sparsity regularization loss, Eₘₐₛₖ mask initialization loss, E_{flow-recon} flow reconstruction loss, and E_{alpha-warp} is the temporal consistency term to the alpha mattes. The λₓ parameters are used as weighting coefficients for the loss function. Details of each parameter of this loss function can be found at Lu et al. [77]. Equation 1 shows the necessary steps required for layer separation. Again, FIG. 1C is a graphical representation of Equation 1 as Equation 2).

Algorithm 1 Layer Separation
Input: XCA Data Matrix, Iₜ

| 1: | Process: | |
|---|---|---|
| 2: | Mₜ ← mask_generator(Iₜ) | [symbol] Section 2.2.1 |
| 3: | Fₜ ← forward_optical flow(Iₜ) | [symbol] Section 2.2.2 |
| 4: | Hₜ ← motion_registration(Iₜ) | [symbol] Section 2.2.3 |
| 5: | while not converged do | |
| 6: | ${C}_{t}^{i} ← Omnimatte ( {I}_{t} , {H}_{t} , {M}_{t}^{i} , {F}_{t}^{i} ) ⊳ {C}_{t}^{i} contains$ | |
| | both the foreground and background layers | |
| 7: | end while | |

$Output : Both foreground and background layers , {C}_{t}^{i}$

### Mask Generation.

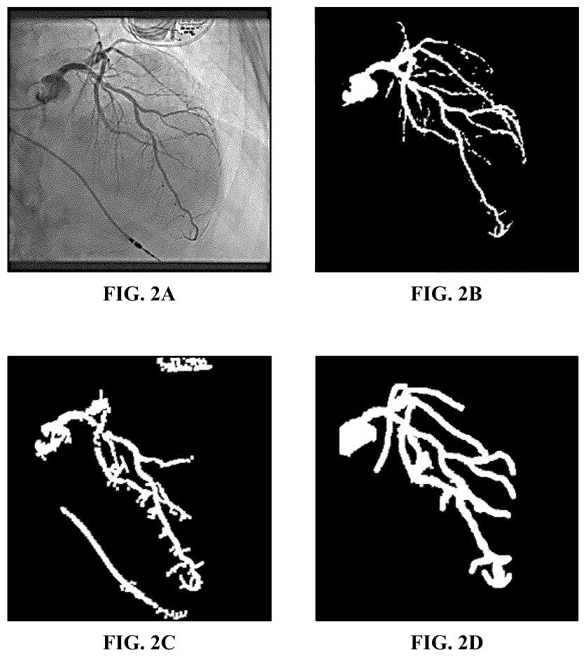

As disclosed herein, a rough mask of the vessels for each frame in the XCA sequence needs to be generated as shown in FIG. 1C. A rough mask may be generated using one of the off-the-shelf classic algorithms such as RPCA [5, 16, 47], MCR-RPCA [5], TV-RPCA [3], Unrolled RPCA [16], MoG-RPCA or deep learning algorithms like UNet [55], UTNet [59], UNet-R [60], UNet++ [61], etc. One can also generate a mask using custom mask-generation tools. In one example, a simple GUI based mask generator/corrector tool was constructed for this purpose which incorporated a custom mask drawing tool, a simple implementation of RPCA [16], and a variation of β-VAE [79]. As shown in FIGS. 2A-D, masks may be generated by different methods as disclosed herein. FIG. 2A shows an original XCA image, 2B illustrates a VAE Generated Mask, 2C illustrates a robust-PCA generated mask, and 2D illustrates a hand-drawn mask.

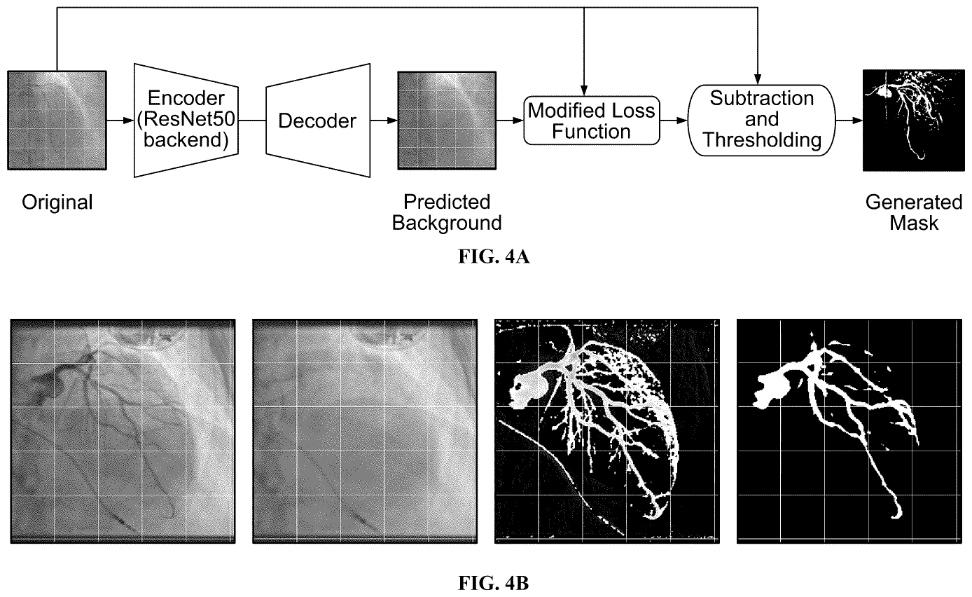

FIGS. 4A-4B show a few ways to generate masks in accordance with the methods and systems disclosed herein. In one example, a variation of the β-VAE-based model was used to generate a rough mask of the vessel layers. No additional erosion or dilation techniques were utilized as a post-processing step for these generated masks. In future implementations, post-processing steps with the mask generation model may be included. RPCA-based models also picked up artifacts, see FIG. 2A middle upper edge, in the mask. The β-VAE model, however, did not pick up these artifacts, thus making it more robust for generating masks on XCA images.

### Optical Flow Generation.

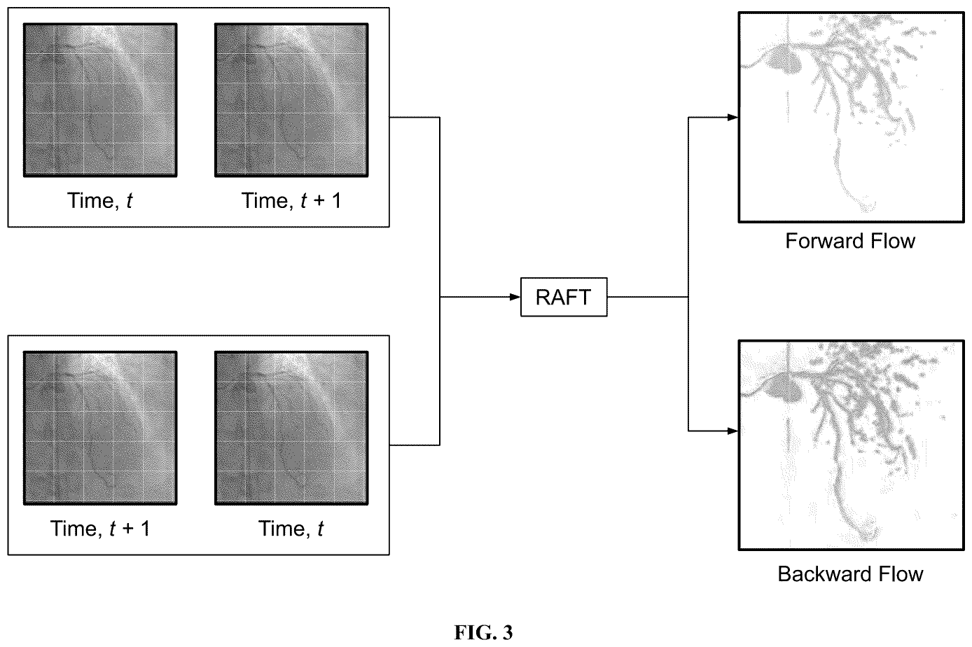

To integrate the model with explicit motion information of the vessel and frame-to-frame correspondence, a dense optical flow field is incorporated. The draft mask may be used to mask the flow information to extract the flow information of the vessel regions only. To estimate the per-pixel motion, i.e., optical flow between XCA frames, a RAFT algorithm was utilized as shown in FIG. 3. Two separate optical flows were computed, namely, forward flow and backward flow as shown in FIG. 1C. Now, to compute the forward flow the XCA frames are passed in their natural order (i.e., time, t=1, 2, 3, . . . , n) and backward flow is computed by passing the XCA frames in reverse order (i.e., time, t=n, n−1, n−2, . . . , 1). Both forward and backward flow from the XCA sequences was generated, but only forward flow was utilized to inject information about the motion information into the final model and improve layer decomposition. In other examples, the backward flow may be used to inject information about the motion information into the final model and improve layer decomposition. In still other examples, both the forward flow and the backward flow may be used to inject information about the motion information into the final model and improve layer decomposition. To compute the uncertain areas of the computed dense flow, both forward and backward flow were utilized to build a confidence map. The confidence map identifies areas where the optical flow calculation is less confident due to factors such as occlusions, noise, or sudden changes in motion. This provides the layer separation network/model with additional information on the motion uncertainty for the dense optical flow.

### Motion Registration.

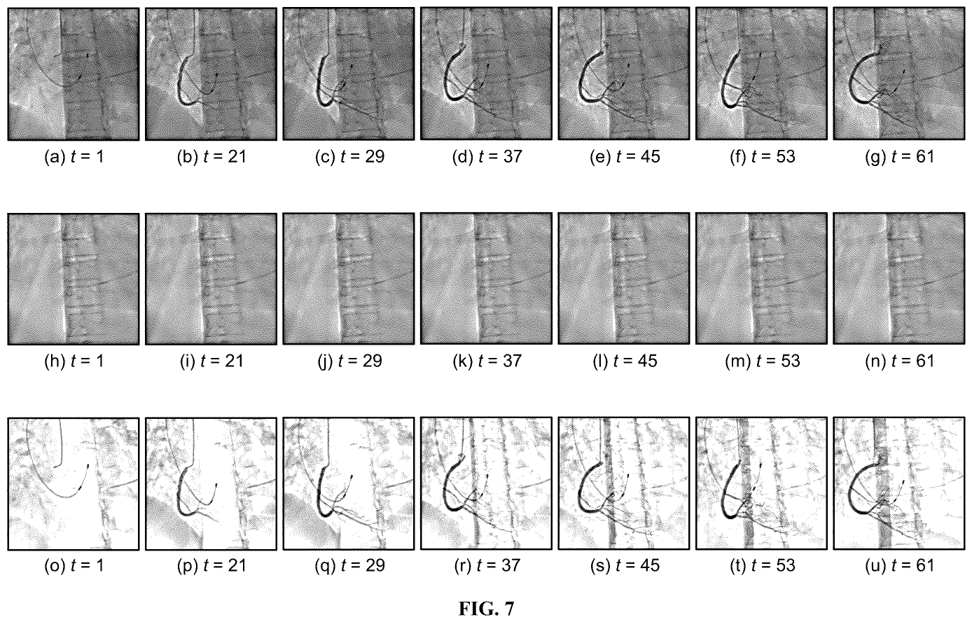

One of the important assumptions for Omnimatte is that the model assumes the background scene is stationary and the foreground can be assumed to be that of slow cardiac motion. With respect to the static background, the foreground motion can be captured by a time-varying homography [81]. A tracker may be used to record the features across all the XCA frames. Minor stability errors may appear in the separated layer, but are usually minor unless large motions are recorded in the static background. For example, FIG. 7 illustrates a case where there is a large motion in the background. Thus, the separated layer captures the motion artifacts, and part of the spine-bone present in the XCA image can be seen in the foreground layer in FIG. 7.

### Layer Separation.

Motivated by B-VAE [82], this model was used to reconstruct the static background of the XCA (which has both static background and vessel layer) image and, as a second objective, to generate masks for the vessel layer. It was assumed that for any given input XCA image x⁽ⁱ⁾, it was desirable to reconstruct the input \acute{x}⁽ⁱ⁾ such that it was the static background of the original XCA. Thus, taking the x⁽ⁱ⁾−\acute{x}⁽ⁱ⁾ will generate the vessel layer. This was done by modeling x⁽ⁱ⁾ into latent space z∈R^{d}. In this case, p(z) is the generative model that that generates the static background and is defined by Gaussian prior N(0, I). The decoder was parameterized by a neural network p_{θ}(x⁽ⁱ⁾z). The encoder is defined by

$${q}_{θ} ( z \vert x ) = {Π}_{j = 1}^{d} N ( {z}_{j} \vert {μ}_{i} ( {x}^{( i )} ) , {σ}_{j}^{2} ( {x}^{( i )} ) ) .$$

The mean and variance may be generated by the encoder q_{θ}(z|x), which is also parameterized by a neural net. The system disclosed herein was constructed with the neural net for q_{θ}(z|x) using a ResNet50 backend. Thus, the variational part of the model (as defined by β-VAE) is defined as E_{q(z|x}⁽ⁱ⁾₎ [log p(x⁽ⁱ⁾|z)]−βKL(q(z|x⁽ⁱ⁾)∥p (z)). Additionally, Dice loss [83] was added as an additional loss to calculate the similarity between images. Dice coefficient may be used to align each pixel between input and predicted images. The sigmoid of both images was taken to achieve this goal. Adding Dice loss functions as a regularizer to the original loss function. The effect of Dice loss is controlled by adding a multiplying factor, γ. To incorporate the image's characteristic features with respect to the generated image, the Structural Similarity Index Matrix (SSIM) [84] is introduced. The SSIM Index considers luminance, contrast, and structural similarity when computed. It is a local measure that considers that the human visual system can only perceive a local area at high resolution at any given time rather than a global measure. The effect of SSIM loss is controlled by adding a multiplying factor, δ. Thus, the final loss function is constructed as Equation 5:

$$ℒ ( x , \hat{x} ) := [ {\sum }_{i = 1}^{N} [ {𝔼}_{q ( z \vert {x}^{( i )} )} [ log p ( {x}^{( i )} \vert z ) ] - β KL ( q ( z \vert {x}^{( i )} ) ‖ p ( z ) ) ] + γ {\sum }_{i = 1}^{N} DiceLoss ( {x}^{( i )} , {\hat{x}}^{( i )} ) + δ \frac{1}{N} {\sum }_{i = 1}^{N} SSIM ( {x}^{( i )} , {\hat{x}}^{( i )} ) ] \qquad ( 5 )$$

Equation 6 is arrived at by expanding DiceLoss (x, \acute{x}) and SSIM (x, \acute{x}). A modified version of DiceLoss(x, \acute{x}) may be utilized. Dice Loss usually takes mask images with values ranging between 0 and 1. But in this case, there is no initial mask; rather, the input image is transformed using a sigmoid function (S(·)). Thus, it can be written as Equation 6:

$$ℒ ( x , \hat{x} ) := [ {\sum }_{i = 1}^{N} [ {𝔼}_{q ( z \vert {x}^{( i )} )} [ log p ( {x}^{( i )} \vert z ) ] - β KL ( q ( z \vert {x}^{( i )} ) ‖ p ( z ) ) ] + γ [ 1 - \frac{2 {\sum }_{i = 1}^{N} 𝒮 ( {x}^{( i )} ) 𝒮 ( {\hat{x}}^{( i )} )}{{\sum }_{i = 1}^{N} 𝒮 ( {x}^{( i )} ) {\sum }_{i = 1}^{N} 𝒮 ( {\hat{x}}^{( i )} )} ] + δ \frac{1}{N} {\sum }_{i = 1}^{N} \frac{( 2 {μ}_{{x}^{( i )}} {μ}_{{\hat{x}}^{( i )}} + {C}_{1} ) ( 2 {σ}_{{x}^{( i )} {\hat{x}}^{( i )}} + {C}_{2} )}{( {μ}_{{x}^{( i )}}^{2} + {μ}_{{\hat{x}}^{( i )}}^{2} + {C}_{1} ) ( {σ}_{{x}^{( i )}}^{2} + {σ}_{{\hat{x}}^{( i )}}^{2} + {C}_{2} )} ] \qquad ( 6 )$$

In Equation 6, μₓ represents the luminance, σₓ represents the contrast,

$$\frac{x - {u}_{x}}{{σ}_{x}}$$

represents the structure of an input image, x. Both l and C may be used to compute luminance and contrast comparison functions between two images. The details of these functions can be found in Wang et al. [84]. The hyper-parameters δ, γ, δ are optimized by grid-search approach. The optimal values calculated for the system and method disclosed herein for generating the background (and subsequently the mask) were β=−0.005, γ=0.5, and δ=1 (The optimization steps and convergence information are not included herein).

FIG. 4A illustrates the overall architecture of the method disclosed herein. FIG. 4B illustrates an end-to-end example of the images generated by the system and method disclosed herein. The first column of FIG. 4B shows the input XCA image, and the second column is the predicted/generated static background. By the convention of digital subtraction angiogram, the predicted background is subtracted from the original XCA image to generate the subtracted image (the third column). By using the appropriate threshold, the mask is generated for the original XCA (the last column). The model was pre-trained with labeled neuro angiogram data from a dataset of cardio cases, and fine-tuned using the cardio data to generate masks.

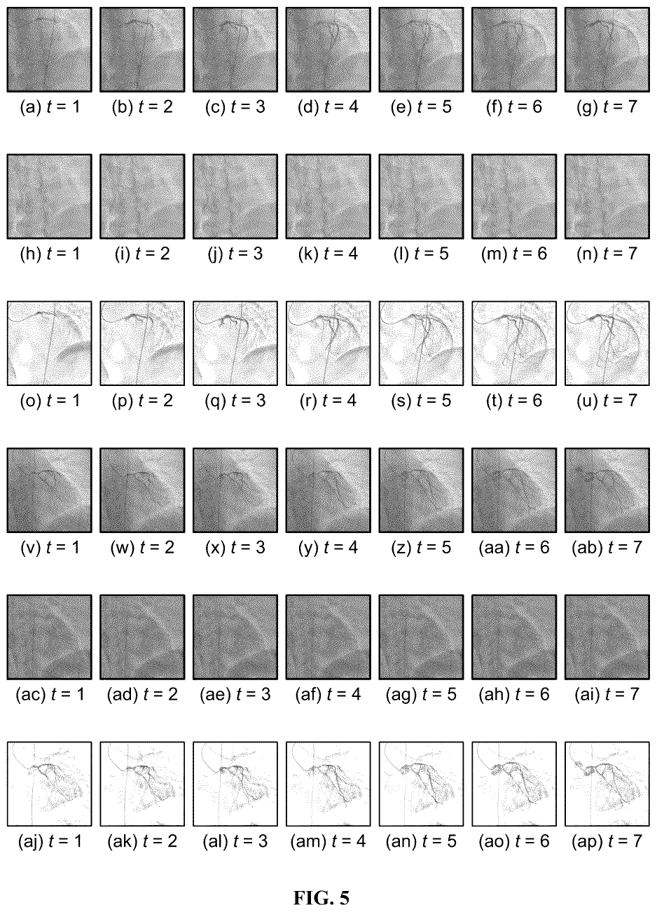

FIG. 5 illustrates two separate examples of estimated foreground and background layers generated from real XCA input sequences using Algorithm/Equation 1. For the selected XCA frames in FIG. 5 (a-g), the stationary background layers can be observed in FIG. 5 (h-n), which have been extracted from the original XCA frames. As there was no large movement in the raw XCA images, the separated background (or foreground) did not have any motion artifacts. A similar observation applies to the separated foreground vessel layer shown in FIG. 5 (o-u). The extracted vessel layer images retained all the information (including minor vessels) present in the raw XCA images.

A similar observation can be made for selected XCA frames in FIG. 5 (v-ab), the stationary background layers can be observed in FIG. 5 (ac-ai), and foreground vessel layer is shown in FIG. 5 (aj-ap).

```
Algorithm 2 Post-processing the Foreground Layer
Input:
  1: Foreground Layer of Input XCA, I_{f}ⁱ
  2: Associated Mask, Mₓⁱ
  3: Noise threshold, tr [symbol]   Pixels of blobs in mask
  4: Initialize → kernel, k
  5: Ifₑᵣⁱ ← eroded_mask (Mₓⁱ, k) [symbol]   Erosion of the mask
  6: Ifₑᵣⁱ ← bitwise_XOR (Mₓⁱ, I_{fer}ⁱ)
  7: I_{jdi}ⁱ ← diluted_mask (Mₓⁱ, k) [symbol]   Dilution of the mask
  8: I_{jdi}ⁱ ← bitwise_XOR (Mₓⁱ, I_{fer}ⁱ)
  9: Iₜᵣᵢₘₐₚⁱ ← add (Mₓⁱ, I_{fer}ⁱ ,I_{jdi}ⁱ) [symbol]   Adding both masks
    together
  10: if Iₜᵣᵢₘₐₚ ≥ tr then
  11: Iₜᵣᵢₘₐₙ → 455
  12: else
  13: Pass
  14: end if
  15: Iₜᵣᵢₘₐₚ ← bitwise_OR (I_{f}ⁱ, I_{f}ⁱ, mask = Iₜᵣᵢₘₐₚ) [symbol]
    masked Bitwise OR Operation
Output: Iₜᵣᵢₘₐₚ
```

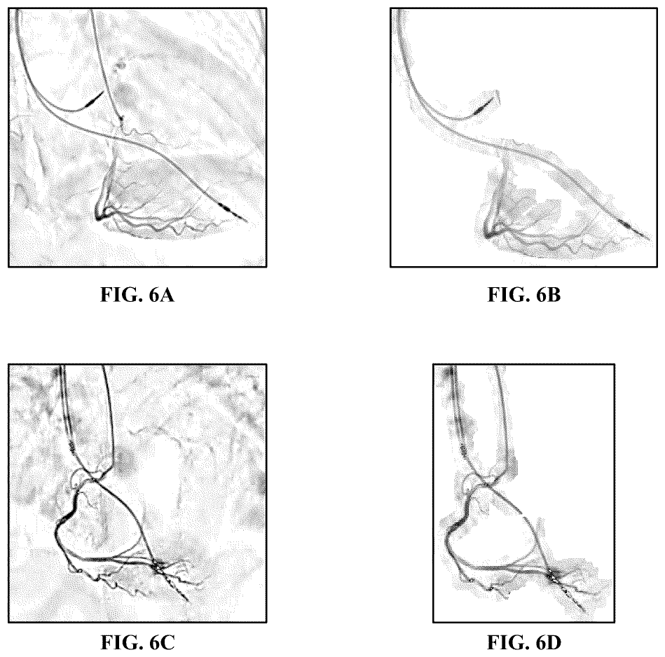

An optional additional step was also developed for post-processing the foreground layer of the original XCA image. The purpose of this step is to remove any additional layer features that may hinder the visuals of XCA vessels. For example, FIGS. 6A and 6C show two extracted foreground layers using proposed Algorithm/Equation 1. FIG. 6A illustrates extracted additional muscle movements that are visible in the bottom left corner. The movement of the diaphragm is also visible in the top right corner. A similar case can also be seen in FIG. 6C. Thus, to have a cleaner visual, a post-processing algorithm to remove additional layer features except for the vessel features was proposed. The pseudo-code for post-processing in Algorithm 2 is shown for this step. For this step, it is required that the rough masks generated in the first step of the model and the foreground layer are generated by using Algorithm 1. A threshold may also be defined to remove any artifacts that are present in the mask. The initial mask may be used to generate trimap

$$( {I}_{ftrimap}^{i} )$$

by first generating an eroded

$$( ( {I}_{fer}^{i} )$$

and diluted

$$( {I}_{fdi}^{i} )$$

mask. The trimap is generated to identify the possible area of influence and uncertain areas where the vessel layers might exist. Next, the threshold (tr) may be used to remove any disconnected pixels generated due to the above process. (Methods like Radon-like Filters [85], or Otsu's global thresholding techniques can also be used to remove unwanted pixels from the map). A bitwise XOR operation on the foreground layer on the masked area (the trimap mask) was then performed to generate the final processed layer, as shown in FIGS. 6B and 6D. While the post-processed image removes all the artifacts, the adjacent vessel layer information is retained to give the vessel relevant context. By adjusting the kernel size, the context area adjacent to the vessel area can be increased.

The proposed model assumes no (or low) movement in the background layer of the XCA images. Also, a dense optical flow is utilized to identify relevant pixels that move at the same frequency. Thus, if there is a significant movement in the background layers, the dense optical flow may pick up the background artifacts (bones, tissues, etc.). FIG. 7 illustrates such an example where, within the XCA image sequence, the spine had significant movement, and that was recognized by the model as seen in FIG. 7 (o-u). The movement of the spine can be noticed starting at t=37 sec, and the movement ends at t=61 sec. Due to this movement, the background layer reconstruction has some blurry reconstruction (notice the spine on the bottom right corner). These artifacts, due to movement, are also visible in the foreground layer (the artifacts are visible from t=37 secs and onward).

While the proposed model does an excellent job of extracting the foreground vessel layers with contextual information, this approach may have certain limitations. The proposed model may have difficulty extracting highly intricate and/or small detailed structures (e.g., small vessels). The model's performance may be sensitive to noise present in the input XCA sequences and may potentially lead to imprecise matte generation. As previously discussed, the model may struggle to accurately extract matte for moving objects in the presence of motion blur or large panning. Finally, the proposed model's computational complexity may hinder real-time or near real-time processing and present challenges when in need of immediate outcomes. As future steps to address the limitations identified in the disclosed method and system, it is planned to further improve the proposed method and systems by incorporating a wide range of techniques ranging from the incorporation of pre-processing methods to mitigate the impact of noise to developing a stabilization algorithm (incorporating temporal information or motion estimation) to overcome issues related to motion artifacts. To overcome computational complexity, it is planned to utilize the processed data generated from the proposed model to train a supervised model similar to the model previously disclosed herein to perform online inference and facilitate real-time processing, and to accommodate larger new datasets.

Disclosed herein is a self-supervised deep learning pipeline to facilitate the segmentation of blood vessels from X-ray coronary angiography image sequences. The systems and methods disclosed herein achieved unexpected results by overcoming the limitations of existing vessel extraction techniques by utilizing low-dimensional latent space features and a video layer separation algorithm to separate the XCA image sequences into the stationary background and dynamic foreground images. The systems and methods disclosed herein were demonstrated as robust, as efficient, and computationally feasible, making the systems and methods promising tools overcoming many failed attempts by industry and the attempts of others to overcome known limitations of existing vessel extraction techniques. By leveraging the self-supervised nature of the proposed method, the need for labeling a large amount of data was bypassed, which is typically required for training deep learning models. In addition, the methods and systems disclosed herein may be used to generate more diverse datasets to ensure its applicability in different aspects of human anatomy. The systems and methods disclosed herein hold great potential for improving the diagnosis and treatment of cardiovascular diseases, ultimately contributing to better patient outcomes and more efficient healthcare delivery. By generating an improved angiographic image comprising recovered vessel structures via the methods and systems disclosed herein, physicians can better diagnose and prescribe a treatment plan for individuals with cardiac related diseases.

### REFERENCES

[1] WHO, World health statistics 2022: monitoring health for the SDGs, sustainable development goals, 2022. URL: https://www.who.int/publications-detail-redirect/9789240051157.
[2] N. Townsend, L. Wilson, P. Bhatnagar, K. Wickramasinghe, M. Rayner, M. Nichols, Cardiovascular disease in Europe: epidemi-ological update 2016, European Heart Journal 37 (2016) 3232-3245.
[3] Z. Fu, Z. Fu, C. Lu, J. Yan, J. Fei, H. Han, Robust Implementation of Foreground Extraction and Vessel Segmentation for X-ray Coronary Angiography Image Sequence, 2022. URL: http://arxiv.org/abs/2209.07237. doi: 10.48550/arXiv.2209.07237, arXiv: 2209.07237 [cs].
[4] H. Rafii-Tari, C. J. Payne, G.-Z. Yang, Current and Emerging Robot-Assisted Endovascular Catheterization Technologies: A Review, An-nals of Biomedical Engineering 42 (2014) 697-715.
[5] M. Jin, R. Li, J. Jiang, B. Qin, Extracting contrast-filled vessels in X-ray angiography by graduated RPCA with motion coherency constraint, Pattern Recognition 63 (2017) 653-666.
[6] S. Albarqouni, J. Fotouhi, N. Navab, X-Ray In-Depth Decomposition: Revealing the Latent Structures, in: M. Descoteaux, L. Maier-Hein, A. Franz, P. Jannin, D. L. Collins, S. Duchesne (Eds.), Medical Image Computing and Computer Assisted Intervention MICCAI 2017, Lec-ture Notes in Computer Science, Springer International Publishing, Cham, 2017, pp. 444-452. doi: 10.1007/978-3-319-66179-7_51.
[7] J. Yang, Y. Wang, Y. Liu, S. Tang, W. Chen, Novel Approach for 3-D Reconstruction of Coronary Arteries From Two Uncalibrated Angiographic Images, IEEE Transactions on Image Processing 18 (2009) 1563-1572. Conference Name: IEEE Transactions on Image Processing.
[8] J. Guo, Y. Cheng, S. Guo, W. Du, A novel path planning algorithm for the vascular interventional surgical robotic doctor training system, in: 2017 IEEE International Conference on Mechatronics and Automa-tion (ICMA), 2017, pp. 45-50. doi: 10.1109/ICMA.2017.8015786, ISSN: 2152-744X.
[9] C. B. Compas, T. Syeda-Mahmood, P. McNeillie, D. Beymer, Au-tomatic detection of coronary stenosis in X-ray angiography through spatio-temporal tracking, in: 2014 IEEE 11th International Sympo-sium on Biomedical Imaging (ISBI), 2014, pp. 1299-1302. doi: 10. 1109/ISBI.2014.6868115, ISSN: 1945-8452.
[10] C. Sui, Z. Fu, Z. Fu, Y. Wang, Y. Zhuang, R. Xie, Y. Zhao, J. Zhang, J. Fei, A Novel Method for Vessel Segmentation and Automatic Diagnosis of Vascular Stenosis, in: 2019 IEEE International Con-ference on Robotics and Biomimetics (ROBIO), 2019, pp. 918-923. doi: 10.1109/ROBIO49542.2019.8961632.
[11] D. Toth, M. Panayiotou, A. Brost, J. M. Behar, C. A. Rinaldi, K. S. Rhode, P. Mountney, 3D/2D Registration with superabundant vessel reconstruction for cardiac resynchronization therapy, Medical Image Analysis 42 (2017) 160-172.
[12] H. Ge, S. Ding, D. An, Z. Li, H. Ding, F. Yang, L. Kong, J. Xu, J. Pu, B. He, Frame counting improves the assessment of post-reperfusion microvascular patency by TIMI myocardial perfusion grade: Evidence from cardiac magnetic resonance imaging, International Journal of Cardiology 203 (2016) 360-366.
[13] S. Ding, J. Pu, Z.-q. Qiao, P. Shan, W. Song, Y. Du, J.-Y. Shen, S.-x. Jin, Y. Sun, L. Shen, Y.-1. Lim, B. He, TIMI myocardial perfusion frame count: A new method to assess myocardial perfu-sion and its predictive value for short-term prognosis, Catheteriza-tion and Cardiovascular Interventions 75 (2010) 722-732._eprint: https://onlinelibrary.wiley.com/doi/pdf/10.1002/ccd.22298.
[14] Z. Fu, Z. Fu, Z. Gong, X. Feng, H. Gu, R. Xie, J. Zhang, J. Fei, Optimization For 3D Reconstruction Of Coronary Artery Tree By Two-stage Levenberg-Marquardt Algorithm, in: 2021 27th Interna-tional Conference on Mechatronics and Machine Vision in Practice (M2VIP), 2021, pp. 84-89. doi: 10.1109/M2VIP49856.2021.9665150.
[15] H. C. Lowe, S. N. Oesterle, L. M. Khachigian, Coronary in-stent restenosis: Current status and future strategies, Journal of the Amer-ican College of Cardiology 39 (2002) 183-193. Publisher: American College of Cardiology Foundation.
[16] B. Qin, H. Mao, Y. Liu, J. Zhao, Y. Lv, Y. Zhu, S. Ding, X. Chen, Robust PCA Unrolling Network for Super-Resolution Vessel Extrac-tion in X-Ray Coronary Angiography, IEEE Transactions on Medical Imaging 41 (2022) 3087-3098. Conference Name: IEEE Transactions on Medical Imaging.
[17] P. Irrera, I. Bloch, M. Delplanque, A flexible patch based approach for combined denoising and contrast enhancement of digital X-ray images, Medical Image Analysis 28 (2016) 33-45.
[18] A. Dogra, B. Goyal, S. Agrawal, Osseous and digital subtraction angiography image fusion via various enhancement schemes and Laplacian pyramid transformations, Future Generation Computer Systems 82 (2018) 149-157.
[19] W. D. Jeans, The development and use of digital subtraction angiogra-phy, The British Journal of Radiology 63 (1990) 161-168. Publisher: The British Institute of Radiology.
[20] D. Podlesek, T. Meyer, U. Morgenstern, G. Schackert, M. Kirsch, Improved Visualization of Intracranial Vessels with Intraoperative Coregistration of Rotational Digital Subtraction Angiography and Intraoperative 3D Ultrasound, PLOS ONE 10 (2015) e0121345. Publisher: Public Library of Science.
[21] M. Saljoughian, Intravenous Radiocontrast Media: A Review of Al-lergic Reactions, 2012. URL: https://www.uspharmacist.com/article/intravenous-radiocontrast-media-a-review-of-allergic-reactions.
[22] M. J. Cha, D. Y. Kang, W. Lee, S. H. Yoon, Y. H. Choi, J. S. Byun, J. Lee, Y.-H. Kim, K. S. Choo, B. S. Cho, K. N. Jeon, J.-W. Jung, H.-R. Kang, Hypersensitivity Reactions to Iodinated Contrast Media: A Multicenter Study of 196 081 Patients, Radiology 293 (2019) 117-124. Publisher: Radiological Society of North America.
[23] S. Xia, H. Zhu, X. Liu, M. Gong, X. Huang, L. Xu, H. Zhang, J. Guo, Vessel Segmentation of X-Ray Coronary Angiographic Image Sequence, IEEE Transactions on Biomedical Engineering 67 (2020) 1338-1348. Conference Name: IEEE Transactions on Biomedical Engineering.
[24] S. çimen, A. Gooya, M. Grass, A. F. Frangi, Reconstruction of coronary arteries from X-ray angiography: A review, Medical Image Analysis 32 (2016) 46-68.
[25] C. T. Metz, M. Schaap, S. Klein, N. Baka, L. A. Neefjes, C. J. Schultz, W. J. Niessen, T. van Walsum, Registration of $3\rm D+\rm t$ Coronary CTA and Monoplane $2\rm D+\rm t$ X-Ray Angiography, IEEE Transactions on Medical Imaging 32 (2013) 919-931. Conference Name: IEEE Transactions on Medical Imaging.
[26] M. Nejati, H. Pourghassem, Multiresolution Image Registration in Digital X-Ray Angiography with Intensity Variation Modeling, Journal of Medical Systems 38 (2014) 10.
[27] M. Nejati, S. Sadri, R. Amirfattahi, Nonrigid Image Registration in Digital Subtraction Angiography Using Multilevel B-Spline, BioMed Research International 2013 (2013) e236315. Publisher: Hindawi.
[28] Y. Deuerling-Zheng, M. Lell, A. Galant, J. Hornegger, Motion com-pensation in digital subtraction angiography using graphics hardware, Computerized Medical Imaging and Graphics 30 (2006) 279-289.
[29] H. Ma, A. Hoogendoorn, E. Regar, W. J. Niessen, T. van Walsum, Automatic online layer separation for vessel enhancement in X-ray angiograms for percutaneous coronary interventions, Medical Image Analysis 39 (2017) 145-161.
[30] M. Hemmendorff, H. Knutsson, M. T. Andersson, T. Kronander, Motion-compensated digital subraction angiography, in: Medical Imaging 1999: Image Processing, volume 3661, SPIE, 1999, pp. 1396-1405. URL: https://www.spiedigitallibrary.org/conference-proceedings-of-spie/3661/0000/Motion-compensated-digital-subraction-angiography/10. 1117/12.348538.full. doi: 10.1117/12.348538.
[31] S. Song, C. Du, Y. Chen, D. Ai, H. Song, Y. Huang, Y. Wang, J. Yang, Inter/intra-frame constrained vascular segmentation in X-ray angiographic image sequence, BMC Medical Informatics and Decision Making 19 (2019) 1-11. Number: 6 Publisher: BioMed Central.
[32] F. Azizmohammadi, R. Martin, J. Miro, L. Duong, Model-free Car-diorespiratory Motion Prediction from X-ray Angiography Sequence with LSTM Network, in: 2019 41st Annual International Conference of the IEEE Engineering in Medicine and Biology Society (EMBC), 2019, pp. 7014-7018. doi: 10.1109/EMBC.2019.8857798, ISSN: 1558-4615.
[33] I. Cruz-Aceves, F. Cervantes-Sanchez, M. S. Avila-Garcia, A Novel Multiscale Gaussian-Matched Filter Using Neural Networks for the Segmentation of X-Ray Coronary Angiograms, Journal of Healthcare Engineering 2018 (2018) e5812059. Publisher: Hindawi.
[34] T. Jerman, F. Pernuš, B. Likar, Špiclin, Blob Enhancement and Visualization for Improved Intracranial Aneurysm Detection, IEEE Transactions on Visualization and Computer Graphics 22 (2016) 1705-1717. Conference Name: IEEE Transactions on Visualization and Computer Graphics.
[35] T. Jerman, F. Pernuš, B. Likar, Špiclin, Enhancement of Vascular Structures in 3D and 2D Angiographic Images, IEEE Transactions on Medical Imaging 35 (2016) 2107-2118. Conference Name: IEEE Transactions on Medical Imaging.
[36] A. F. Frangi, W. J. Niessen, K. L. Vincken, M. A. Viergever, Mul-tiscale vessel enhancement filtering, in: W. M. Wells, A. Colch-ester, S. Delp (Eds.), Medical Image Computing and Computer—Assisted Intervention—MICCAI'98, Lecture Notes in Computer Science, Springer, Berlin, Heidelberg, 1998, pp. 130-137. doi: 10. 1007/BFb0056195.
[37] Y. Sato, S. Nakajima, N. Shiraga, H. Atsumi, S. Yoshida, T. Koller, G. Gerig, R. Kikinis, Three-dimensional multi-scale line filter for segmentation and visualization of curvilinear structures in medical images, Medical Image Analysis 2 (1998) 143-168.
[38] W. Zhang, H. Ling, S. Prummer, K. S. Zhou, M. Ostermeier, D. Co-maniciu, Coronary Tree Extraction Using Motion Layer Separation, in: G.-Z. Yang, D. Hawkes, D. Rueckert, A. Noble, C. Taylor (Eds.), Medical Image Computing and Computer-Assisted Intervention-MICCAI 2009, Lecture Notes in Computer Science, Springer, Berlin, Heidelberg, 2009, pp. 116-123. doi: 10.1007/978-3-642-04268-3_15.
[39] Y. Zhu, S. Prummer, P. Wang, T. Chen, D. Comaniciu, M. Ostermeier, Dynamic Layer Separation for Coronary DSA and Enhancement in Fluoroscopic Sequences, in: G.-Z. Yang, D. Hawkes, D. Rueckert, A. Noble, C. Taylor (Eds.), Medical Image Computing and Computer-Assisted Intervention-MICCAI 2009, Lecture Notes in Computer Science, Springer, Berlin, Heidelberg, 2009, pp. 877-884. doi: 10. 1007/978-3-642-04271-3_106.
[40] J. S. Preston, C. Rottman, A. Cheryauka, L. Anderton, R. T. Whitaker, S. Joshi, Multi-layer Deformation Estimation for Fluoroscopic Imag-ing, in: J. C. Gee, S. Joshi, K. M. Pohl, W. M. Wells, L. Zöllei (Eds.), Information Processing in Medical Imaging, Lecture Notes in Computer Science, Springer, Berlin, Heidelberg, 2013, pp. 123-134. doi: 10.1007/978-3-642-38868-2_11.
[41] S. Tang, Y. Wang, Y.-W. Chen, Application of ICA to X-ray coronary digital subtraction angiography, Neurocomputing 79 (2012) 168-172.
[42] B. Qin, Z. Gu, X. Sun, Y. Lv, Registration of Images With Outliers Using Joint Saliency Map, IEEE Signal Processing Letters 17 (2010) 91-94. Conference Name: IEEE Signal Processing Letters.
[43] G. Wang, Y. Chen, X. Zheng, Gaussian field consensus: A robust non-parametric matching method for outlier rejection, Pattern Recognition 74 (2018) 305-316.
[44] B. Qin, Z. Shen, Z. Zhou, J. Zhou, Y. Lv, Structure matching driven by joint-saliency-structure adaptive kernel regression, Applied Soft Computing 46 (2016) 851-867.
[45] S. Zhang, K. Yang, Y. Yang, Y. Luo, Z. Wei, Non-rigid point set registration using dual-feature finite mixture model and global-local structural preservation, Pattern Recognition 80 (2018) 183-195.
[46] B. Qin, Z. Shen, Z. Fu, Z. Zhou, Y. Lv, J. Bao, Joint-Saliency Structure Adaptive Kernel Regression with Adaptive-Scale Kernels for Deformable Registration of Challenging Images, IEEE Access 6 (2018) 330-343. Conference Name: IEEE Access.
[47] Z. Shao, Y. Pu, J. Zhou, B. Wen, Y. Zhang, Hyper RPCA: Joint Max-imum Correntropy Criterion and Laplacian Scale Mixture Modeling on-the-Fly for Moving Object Detection, IEEE Transactions on Mul-timedia 25 (2023) 112-125. Conference Name: IEEE Transactions on Multimedia.
[48] H. Ma, G. Dibildox, J. Banerjee, W. Niessen, C. Schultz, E. Regar, T. van Walsum, Layer Separation for Vessel Enhancement in Interven-tional X-ray Angiograms Using Morphological Filtering and Robust PCA, in: C. A. Linte, Z. Yaniv, P. Fallavollita (Eds.), Augmented Environments for Computer-Assisted Interventions, Lecture Notes in Computer Science, Springer International Publishing, Cham, 2015, pp. 104-113. doi: 10.1007/978-3-319-24601-7_11.
[49] Q. Zhao, D. Meng, Z. Xu, W. Zuo, L. Zhang, Robust Principal Component Analysis with Complex Noise, in: Proceedings of the 31st International Conference on Machine Learning, PMLR, 2014, pp. 55-63. URL: https://proceedings.mlr.press/v32/zhao14.html, ISSN: 1938-7228.
[50] Q. Gao, P. Zhang, W. Xia, D. Xie, X. Gao, D. Tao, Enhanced Tensor RPCA and its Application, IEEE Transactions on Pattern Analysis and Machine Intelligence 43 (2021) 2133-2140. Conference Name: IEEE Transactions on Pattern Analysis and Machine Intelligence.
[51] Q. Xie, Q. Zhao, D. Meng, Z. Xu, Kronecker-Basis-Representation Based Tensor Sparsity and Its Applications to Tensor Recovery, IEEE Transactions on Pattern Analysis and Machine Intelligence 40 (2018) 1888-1902. Conference Name: IEEE Transactions on Pattern Analysis and Machine Intelligence.
[52] C. Lu, J. Feng, Y. Chen, W. Liu, Z. Lin, S. Yan, Tensor Robust Principal Component Analysis with a New Tensor Nuclear Norm, IEEE Transactions on Pattern Analysis and Machine Intelligence 42 (2020) 925-938. Conference Name: IEEE Transactions on Pattern Analysis and Machine Intelligence.
[53] B. Qin, M. Jin, D. Hao, Y. Lv, Q. Liu, Y. Zhu, S. Ding, J. Zhao, B. Fei, Accurate vessel extraction via tensor completion of background layer in X-ray coronary angiograms, Pattern Recognition 87 (2019) 38-54.
[54] I. Goodfellow, Y. Bengio, A. Courville, Deep Learning, MIT Press, 2016. Google-Books-ID: omivDQAAQBAJ.
[55] O. Ronneberger, P. Fischer, T. Brox, U-Net: Convolutional Networks for Biomedical Image Segmentation, in: N. Navab, J. Hornegger, W. M. Wells, A. F. Frangi (Eds.), Medical Image Computing and Computer-Assisted Intervention-MICCAI 2015, Lecture Notes in Computer Science, Springer International Publishing, Cham, 2015, pp. 234-241. doi: 10.1007/978-3-319-24574-4_28.
[56] çiçek, A. Abdulkadir, S. S. Lienkamp, T. Brox, O. Ronneberger, 3D U-Net: Learning Dense Volumetric Segmentation from Sparse Annotation, in: S. Ourselin, L. Joskowicz, M. R. Sabuncu, G. Unal, W. Wells (Eds.), Medical Image Computing and Computer-Assisted Intervention-MICCAI 2016, Lecture Notes in Computer Science, Springer International Publishing, Cham, 2016, pp. 424-432. doi: 10. 1007/978-3-319-46723-8_49.
[57] B. Kayalibay, G. Jensen, P. van der Smagt, CNN-based Segmentation of Medical Imaging Data, 2017. URL: http://arxiv.org/abs/1701. 03056. doi: 10.48550/arXiv.1701.03056, arXiv: 1701.03056 [cs].
[58] F. Isensee, P. Kickingereder, W. Wick, M. Bendszus, K. H. Maier-Hein, Brain Tumor Segmentation and Radiomics Survival Prediction: Contribution to the BRATS 2017 Challenge, in: A. Crimi, S. Bakas, H. Kuijf, B. Menze, M. Reyes (Eds.), Brainlesion: Glioma, Multiple Sclerosis, Stroke and Traumatic Brain Injuries, Lecture Notes in Computer Science, Springer International Publishing, Cham, 2018, pp. 287-297. doi: 10.1007/978-3-319-75238-9_25.
[59] Y. Gao, M. Zhou, D. N. Metaxas, UTNet: A Hybrid Transformer Architecture for Medical Image Segmentation, in: M. de Bruijne, P. C. Cattin, S. Cotin, N. Padoy, S. Speidel, Y. Zheng, C. Es-sert (Eds.), Medical Image Computing and Computer Assisted In-tervention—MICCAI 2021, Lecture Notes in Computer Science, Springer International Publishing, Cham, 2021, pp. 61-71. doi: 10. 1007/978-3-030-87199-4_6.
[60] A. Hatamizadeh, Y. Tang, V. Nath, D. Yang, A. Myronenko, B. Landman, H. R. Roth, D. Xu, UNETR: Transformers Image Segmentation, URL: 2022, pp. 574-584. https://openaccess.thecvf.com/content/WACV2022/html/Hatamizadeh_UNETR_Transfor mers_for_3D_Medical_Image_Segmentation_WACV_2022_paper.html.
[61] Z. Zhou, M. M. Rahman Siddiquee, N. Tajbakhsh, J. Liang, UNet++: A Nested U-Net Architecture for Medical Image Segmentation, in: D. Stoyanov, Z. Taylor, G. Carneiro, T. Syeda-Mahmood, A. Martel, L. Maier-Hein, J. M. R. Tavares, A. Bradley, J. P. Papa, V. Belagiannis, J. C. Nascimento, Z. Lu, S. Conjeti, M. Moradi, H. Greenspan, A. Madabhushi (Eds.), Deep Learning in Medical Image Analysis and Multimodal Learning for Clinical Decision Support, Lecture Notes in Computer Science, Springer International Publishing, Cham, 2018, pp. 3-11. doi: 10.1007/978-3-030-00889-5_1.
[62] H. Cao, Y. Wang, J. Chen, D. Jiang, X. Zhang, Q. Tian, M. Wang, Swin-Unet: Unet-Like Pure Transformer for Medical Image Seg-mentation, in: L. Karlinsky, T. Michaeli, K. Nishino (Eds.), Com-puter Vision-ECCV 2022 Workshops, Lecture Notes in Computer Science, Springer Nature Switzerland, Cham, 2023, pp. 205-218. doi: 10.1007/978-3-031-25066-8_9.
[63] D. G. Ellis, M. R. Aizenberg, Trialing U-Net Training Modifications for Segmenting Gliomas Using Open Source Deep Learning Frame-work, in: A. Crimi, S. Bakas (Eds.), Brainlesion: Glioma, Multiple Sclerosis, Stroke and Traumatic Brain Injuries, Lecture Notes in Computer Science, Springer International Publishing, Cham, 2021, pp. 40-49. doi: 10.1007/978-3-030-72087-2_4.
[64] R. Azad, M. Heidari, M. Shariatnia, E. K. Aghdam, S. Karim-ijafarbigloo, E. Adeli, D. Merhof, TransDeepLab: Convolution-Free Transformer-Based DeepLab v3+ for Medical Image Segmen-tation, in: I. Rekik, E. Adeli, S. H. Park, C. Cintas (Eds.), Predic-tive Intelligence in Medicine, Lecture Notes in Computer Science, Springer Nature Switzerland, Cham, 2022, pp. 91-102. doi: 10.1007/978-3-031-16919-9_9.
[65] F. Yuan, Z. Zhang, Z. Fang, An effective CNN and Transformer complementary network for medical image segmentation, Pattern Recognition 136 (2023) 109228.
[66] J. Chen, Y. He, E. C. Frey, Y. Li, Y. Du, ViT-V-Net: Vision Trans-former for Unsupervised Volumetric Medical Image Registration, 2021. URL: http://arxiv.org/abs/2104.06468. doi: 10.48550/arXiv. 2104.06468, arXiv: 2104.06468 [cs, eess].
[67] H. Wang, S. Xie, L. Lin, Y. Iwamoto, X.-H. Han, Y.-W. Chen, R. Tong, Mixed Transformer U-Net for Medical Image Segmentation, in: ICASSP 2022-2022 IEEE International Conference on Acoustics, Speech and Signal Processing (ICASSP), 2022, pp. 2390-2394. doi: 10.1109/ICASSP43922.2022.9746172, ISSN: 2379-190X.
[68] D. Karimi, S. D. Vasylechko, A. Gholipour, Convolution-Free Medical Image Segmentation Using Transformers, in: M. de Brui-jne, P. C. Cattin, S. Cotin, N. Padoy, S. Speidel, Y. Zheng, C. Es-sert (Eds.), Medical Image Computing and Computer Assisted In-tervention—MICCAI 2021, Lecture Notes in Computer Science, Springer International Publishing, Cham, 2021, pp. 78-88. doi: 10. 1007/978-3-030-87193-2_8.
[69] J. Chen, Y. Lu, Q. Yu, X. Luo, E. Adeli, Y. Wang, L. Lu, A. L. Yuille, Y. Zhou, TransUNet: Transformers Make Strong Encoders for Medical Image Segmentation, 2021. URL: http://arxiv.org/abs/2102.04306. doi: 10.48550/arXiv.2102.04306, arXiv: 2102.04306 [cs].
[70] X. Huang, Z. Deng, D. Li, X. Yuan, MISSFormer: An Effective Medi-cal Image Segmentation Transformer, 2021. URL: http://arxiv.org/abs/2109.07162. doi: 10.48550/arXiv.2109.07162, arXiv: 2109.07162 [cs].
[71] J. M. J. Valanarasu, P. Oza, I. Hacihaliloglu, V. M. Patel, Medical Transformer: Gated Axial-Attention for Medical Image Segmenta-tion, in: M. de Bruijne, P. C. Cattin, S. Cotin, N. Padoy, S. Speidel, Y. Zheng, C. Essert (Eds.), Medical Image Computing and Computer Assisted Intervention—MICCAI 2021, Lecture Notes in Computer Science, Springer International Publishing, Cham, 2021, pp. 36-46. doi: 10.1007/978-3-030-87193-2_4.
[72] C. Wu, Y. Zou, Z. Yang, U-GAN: Generative Adversarial Networks with U-Net for Retinal Vessel Segmentation, in: 2019 14th Interna-tional Conference on Computer Science & Education (ICCSE), 2019, pp. 642-646. doi: 10.1109/ICCSE.2019.8845397, ISSN: 2473-9464.
[73] Y. Gao, Y. Song, X. Yin, W. Wu, L. Zhang, Y. Chen, W. Shi, Deep learning-based digital subtraction angiography image generation, In-ternational Journal of Computer Assisted Radiology and Surgery 14 (2019) 1775-1784.
[74] D. Ueda, Y. Katayama, A. Yamamoto, T. Ichinose, H. Arima, Y. Watanabe, S. L. Walston, H. Tatekawa, H. Takita, T. Honjo, A. Shimazaki, D. Kabata, T. Ichida, T. Goto, Y. Miki, Deep Learn-ing-based Angiogram Generation Model for Cerebral Angiography without Misregistration Artifacts, Radiology 299 (2021) 675-681. Publisher: Radiological Society of North America.
[75] R. Kimura, A. Teramoto, T. Ohno, K. Saito, H. Fujita, Virtual digital subtraction angiography using multizone patch-based U-Net, Physical and Engineering Sciences in Medicine 43 (2020) 1305-1315.
[76] L. N. L. Thuy, T. D. Trinh, L. H. Anh, J. Y. Kim, H. T. Hieu, P. T. Bao, Coronary Vessel Segmentation by Coarse-to-Fine Strategy Using U-nets, BioMed Research International 2021 (2021) 1-10.
[77] E. Lu, F. Cole, T. Dekel, A. Zisserman, W. T. Freeman, M. Rubin-stein, Omnimatte: Associating Objects and Their Effects in Video, 2021. URL: http://arxiv.org/abs/2105.06993. doi: 10.48550/arXiv. 2105.06993, arXiv: 2105.06993 [cs].
[78] E. Lu, F. Cole, T. Dekel, W. Xie, A. Zisserman, D. Salesin, W. T. Freeman, M. Rubinstein, Layered Neural Rendering for Retiming Peo-ple in Video, 2021. URL: http://arxiv.org/abs/2009.07833. doi: 10. 48550/arXiv.2009.07833, arXiv: 2009.07833 [cs].
[79] H. Kim, A. Mnih, Disentangling by Factorising, in: Proceed-ings of the 35th International Conference on Machine Learning, PMLR, 2018, pp. 2649-2658. URL: https://proceedings.mlr.press/v80/kim18b.html, iSSN: 2640-3498.
[80] Z. Teed, J. Deng, RAFT: Recurrent All-Pairs Field Transforms for Optical Flow, 2020. URL: http://arxiv.org/abs/2003.12039. doi: 10. 48550/arXiv.2003.12039, arXiv: 2003.12039 [cs].
[81] E. Rublee, V. Rabaud, K. Konolige, G. Bradski, ORB: An efficient alternative to SIFT or SURF, in: 2011 International Conference on Computer Vision, 2011, pp. 2564-2571. doi: 10.1109/ICCV.2011. 6126544, ISSN: 2380-7504.
[82] I. Higgins, L. Matthey, A. Pal, C. Burgess, X. Glorot, M. Botvinick, S. Mohamed, A. Lerchner, beta-VAE: Learning Basic Visual Con-cepts with a Constrained Variational Framework, 2022. URL: https://openreview.net/forum?id=Sy2fzU9gl.
[83] C. H. Sudre, W. Li, T. Vercauteren, S. Ourselin, M. Jorge Cardoso, Generalised Dice Overlap as a Deep Learning Loss Function for Highly Unbalanced Segmentations, in: M. J. Cardoso, T. Arbel, G. Carneiro, T. Syeda-Mahmood, J. M. R. Tavares, M. Moradi, A. Bradley, H. Greenspan, J. P. Papa, A. Madabhushi, J. C. Nasci-mento, J. S. Cardoso, V. Belagiannis, Z. Lu (Eds.), Deep Learning in Medical Image Analysis and Multimodal Learning for Clinical Decision Support, Lecture Notes in Computer Science, Springer International Publishing, Cham, 2017, pp. 240-248. doi: 10.1007/978-3-319-67558-9_28.
[84] Z. Wang, A. Bovik, H. Sheikh, E. Simoncelli, Image quality assessment: from error visibility to structural similarity, IEEE Transactions on Image Processing 13 (2004) 600-612. Conference Name: IEEE Transactions on Image Processing.
[85] R. Kumar, A. Vázquez-Reina, H. Pfister, Radon-Like features and their application to connectomics, in: 2010 IEEE Computer Society Conference on Computer Vision and Pattern Recognition Work-shops, 2010, pp. 186-193. doi: 10.1109/CVPRW.2010.5543594, ISSN: 2160-7516.
[86] N. Otsu, A Threshold Selection Method from Gray-Level Histograms, IEEE Transactions on Systems, Man, and Cybernetics 9 (1979) 62-66. Conference Name: IEEE Transactions on Systems, Man, and Cybernetics.

What is claimed is:

1. A method of catheter angiography imaging via a deep neural network-based digital subtraction angiography (DSA) algorithm, wherein each of the foregoing steps are performed by a computing device comprising at least one processor, a communication interface, and memory, comprising:
receiving X-ray coronary angiography (XCA) images from an individual;
generating a mask of vessel layers from the XCA images;
calculating an optical flow comprising a forward flow and a backward flow, determining uncertain areas of dense optical flow using the forward flow and the backward flow;
generating a confidence map using the uncertain areas of dense optical flow, wherein the confidence map identifies areas where the optical flow calculation is less confident; and
extracting vessel layers from a background image and a foreground image, wherein the forward flow is used to extract the vessel layers from the background image and the foreground image, wherein the confidence map to extract the vessel layers from the background image and the foreground image, and wherein the background image and the foreground image comprise voluntary motion, respiratory motion, cardiac motion, or other artifact; and
generating a final angiographic image comprising the extracted vessel layers, wherein the XCA images are received and the final angiographic image is generated in real-time or near real-time.

2. A system for generating an angiographic image comprising the method of claim 1.

3. A method of digital subtraction angiography (DSA) imaging, wherein each of the foregoing steps are performed by a computing device comprising at least one processor, a communication interface, and memory, comprising:
receiving X-ray coronary angiography (XCA) images from an individual;
generating a mask of vessel layers from the XCA images;
extracting vessel layers from a background image and a foreground image, wherein the background image and the foreground image comprise voluntary motion, respiratory motion, cardiac motion, or other artifact; and
generating a final angiographic image comprising the extracted vessel layers.

4. The method of claim 3, wherein the XCA images are received and the final angiographic image is generated in real-time or near real-time.

5. The method of claim 3, further comprising calculating an optical flow comprising a forward flow and a backward flow, wherein the forward flow is used to extract the vessel layers from the background image and the foreground image.

6. The method of claim 5, wherein the forward flow and the backward flow comprise vessel region structure data only.

7. The method of claim 5, further comprising determining uncertain areas of dense optical flow using the forward flow and the backward flow.

8. The method of claim 7, further comprising generating a confidence map using the uncertain areas of dense optical flow, wherein the confidence map identifies areas where the optical flow calculation is less confident, and using the confidence map to extract the vessel layers from the background image and the foreground image.

9. The method of claim 3, further comprising registering the background image as stationary.

10. The method of claim 9, further comprising registering the foreground image as that of a slow cardiac motion prior to extracting the vessel layers from the background image and the foreground image.

11. The method of claim 3, wherein the received XCA images are from an individual in a remote location.

12. A system for generating an angiographic image comprising memory storing computer-readable instructions causing the system to:
receive X-ray coronary angiography (XCA) images from an individual;
generate a mask of vessel layers from the XCA images;
extract the vessel layers from a background image and a foreground image, wherein the background image and the foreground image comprise voluntary motion, respiratory motion, cardiac motion, or other artifact; and
generate a final angiographic image comprising the extracted vessel layers.

13. The system of claim 12, wherein the XCA images are received and the final angiographic image is generated in real-time or near real-time.

14. The system of claim 12, further comprising calculating, via an optical flow generator, an optical flow comprising a forward flow and a backward flow, wherein the forward flow is used to extract the vessel layers from the background image and the foreground image.

15. The system of claim 14, wherein the forward flow and the backward flow comprise vessel region structure data only.

16. The system of claim 14, further comprising determining, via the optical flow generator, uncertain areas of dense optical flow using the forward flow and the backward flow.

17. The system of claim 16, further comprising generating, via a confidence generator, a confidence map using the uncertain areas of dense optical flow, wherein the confidence map identifies areas where the optical flow calculation is less confident, and using the confidence map to extract the vessel layers from the background image and the foreground image.

18. The system of claim 12, wherein the received XCA images are from an individual in a remote location.

19. The system of claim 12, wherein the received XCA images are from a historical database.

20. A non-transitory machine-readable medium storing instructions that, when executed by one or more processors, cause the one or more processors to perform steps comprising:
receiving X-ray coronary angiography (XCA) images from an individual;
generating a mask of vessel layers from the XCA images;
extracting the vessel layers from a background image and a foreground image, wherein the background image and the foreground image comprise voluntary motion, respiratory motion, cardiac motion, or other artifact; and
generating a final angiographic image comprising the extracted vessel layers.
